(19) United States
(12) Patent Application Publication
(10) Pub. No.: US 2019/0200871 A1
(43) Pub. Date: Jul. 4, 2019

(54) SYSTEM AND METHOD FOR VITAL SIGNS DETECTION

(71) Applicant: KONINKLIJKE PHILIPS N.V., EINDHOVEN (NL)

(72) Inventor: Gerard DE HAAN, HELMOND (NL)

(21) Appl. No.: 16/301,561

(22) PCT Filed: Jun. 23, 2017
(86) PCT No.: PCT/EP2017/065601

§ 371 Date: Nov. 14, 2018

(30) Foreign Application Priority Data
Jun. 24, 2016 (EP) 16176091.3

(51) Int. Cl. A61B 5/00

(52) U.S. Cl. CPC A61B 5/0075; A61B 5/02433; A61B 5/0077

(57) ABSTRACT

The present invention relates to a system for vital signs detection. The system comprises a radiation source (16) for emitting radiation in a limited wavelength range for illuminating a skin area of a subject and a radiation detector (12), a radiation detector (12, 30, 40) for detecting radiation reflected from a skin area of a subject (1) in response to said illumination, and for generating first and second detector signals, the first detector signal representing radiation (2) reflected from the skin area of a subject in a first wavelength subrange of said limited wavelength range of radiation (3) and the second detector signal representing radiation in a second wavelength sub-range of said limited wavelength range of radiation different from said first wavelength sub-range, and a vital signs detector (14) for detecting a vital sign from a combination of said first and second detector signals by computing the difference between said first and second detector signals.

### FIELD OF THE INVENTION

The present invention relates to a system and method for vital signs detection.

### BACKGROUND OF THE INVENTION

Vital signs of a person, for example the heart rate (HR), the respiration rate (RR) or the arterial blood oxygen saturation, serve as indicators of the current state of a person and as powerful predictors of serious medical events. For this reason, vital signs are extensively monitored in inpatient and outpatient care settings, at home or in further health, leisure and fitness settings.

One way of measuring vital signs is plethysmography. Plethysmography generally refers to the measurement of volume changes of an organ or a body part and in particular to the detection of volume changes due to a cardio-vascular pulse wave traveling through the body of a subject with every heartbeat.

Photoplethysmography (PPG) is an optical measurement technique that evaluates a time-variant change of light reflectance or transmission of an area or volume of interest. PPG is based on the principle that blood absorbs light more than surrounding tissue, so variations in blood volume with every heart beat affect transmission or reflectance correspondingly. Besides information about the heart rate, a PPG waveform can comprise information attributable to further physiological phenomena such as the respiration. By evaluating the transmittance and/or reflectivity at different wavelengths (typically red and infrared), the blood oxygen saturation can be determined.

Conventional pulse oximeters (also called contact PPG device herein) for measuring the heart rate and the (arterial) blood oxygen saturation (also called SpO2) of a subject are attached to the skin of the subject, for instance to a fingertip, earlobe or forehead. Therefore, they are referred to as ‘contact’ PPG devices. A typical pulse oximeter comprises a red LED and an infrared LED as light sources and one photodiode for detecting light that has been transmitted through patient tissue. Commercially available pulse oximeters quickly switch between measurements at a red and an infrared wavelength and thereby measure the transmittance of the same area or volume of tissue at two different wavelengths. This is referred to as time-division-multiplexing. The transmittance over time at each wavelength gives the PPG waveforms for red and infrared wavelengths. Although contact PPG is regarded as a basically non-invasive technique, contact PPG measurement is often experienced as being unpleasant and obtrusive, since the pulse oximeter is directly attached to the subject and any cables limit the freedom to move and might hinder a workflow. The same holds for contact sensors for respiration measurements, which may sometimes be practically impossible because of extremely sensitive skin (e.g. of patients with burns and preterm infants).

Recently, non-contact, remote PPG (rPPG) devices (also called camera rPPG device herein) for unobtrusive measurements have been introduced. Remote PPG utilizes light sources or, in general radiation sources, disposed remotely from the subject of interest. Similarly, also a detector, e.g., a camera or a photo detector, can be disposed remotely from the subject of interest. Therefore, remote photoplethysmographic systems and devices are considered unobtrusive and well suited for medical as well as non-medical everyday applications. However, remote PPG devices typically achieve a lower signal-to-noise ratio.

Verkruysse et al., “Remote plethysmographic imaging using ambient light”, Optics Express, 16(26), 22 Dec. 2008, pp. 21434-21445 demonstrates that photoplethysmographic signals can be measured remotely using ambient light and a conventional consumer level video camera, using red, green and blue color channels.

Using PPG technology, vital signs can be measured, which are revealed by minute light absorption changes in the skin caused by the pulsating blood volume, i.e. by periodic color changes of the human skin induced by the blood volume pulse. As this signal is very small and hidden in much larger variations due to illumination changes and motion, there is a general interest in improving the fundamentally low signal-to-noise ratio (SNR). There still are demanding situations, with severe motion, challenging environmental illumination conditions, or high required accuracy of the application, where an improved robustness and accuracy of the vital sign measurement devices and methods is required, particularly for the more critical healthcare applications.

To achieve motion robustness, pulse-extraction methods profit from the color variations having an orientation in the normalized RGB color space which differs from the orientation of the most common distortions usually induced by motion. A known method for robust pulse signal extraction uses the known fixed orientation of the blood volume pulse in the normalized RGB color space to eliminate the distortion signals. Further background is disclosed in M. van Gastel, S. Stuijk and G. de Haan, “Motion robust remote-PPG in infrared”, IEEE, Tr. On Biomedical Engineering, Vol. 62, No. 5, 2015, pp. 1425-1433 in G. de Haan and A. van Leest, “Improved motion robustness of remote-PPG by using the blood volume pulse signature”, Physiol. Meas. 35 1913, 2014, which describes that the different absorption spectra of arterial blood and bloodless skin cause the variations to occur along a very specific vector in a normalized RGB-space. The exact vector can be determined for a given light-spectrum and transfer-characteristics of the optical filters in the camera. It is shown that this “signature” can be used to design an rPPG algorithm with a much better motion robustness than the recent methods based on blind source separation, and even better than chrominance-based methods published earlier.

Using cameras in the automotive field for vital signs detection has been considered, but motion robustness in this areas is complicated by the strong requirements to only use the already available NIR (near-infrared) illumination, which originates from a single LED light source (often emitting radiation around 850 nm). The problem is that a camera registering the light reflected from the driver (e.g. the face) cannot distinguish between modulations caused by motion and modulations due to absorption changes of the skin caused by changing blood volume. Although many attempts have been made to solve this issue, to date no satisfactory solution exists.

WO 2015/003938 A1 discloses a processor and a system for screening of the state of oxygenation of a subject, in particular for screening of newborn babies for congenital heart disease. The system comprises an imaging unit for obtaining a plurality of image frames of the subject over time, and a processor for processing the image frames. The imaging unit, for instance a conventional video camera as used in the vital signs monitoring using the above mentioned principle of remote PPG, is used as a contact less pulse oximeter, by use of which a body map (for at least some body parts of interest) of at least the blood oxygen saturation is created. Picking certain body areas, e.g. right upper extremity versus left upper and/or lower extremity, and combining or comparing them can serve the purpose of detecting anomalies of heart and/or circuitry functions.

### SUMMARY OF THE INVENTION

It is an object of the present invention to provide a system and method for motion-robust vital signs detection for use e.g. in the automotive field.

In a first aspect of the present invention a system for vital signs detection is presented comprising:

a radiation source for emitting radiation in a limited wavelength range for illuminating a skin area of a subject,

a radiation detector for detecting radiation reflected from a skin area of a subject in response to said illumination, and for generating first and second detector signals, the first detector signal representing radiation reflected from the skin area of a subject in a first wavelength sub-range of said limited wavelength range of radiation and the second detector signal representing radiation in a second wavelength sub-range of said limited wavelength range of radiation different from said first wavelength sub-range, wherein said radiation detector comprises at least two detector areas, wherein a first detector area is sensitive for radiation in said first wavelength sub-range and is configured to generate said first detector signal and a second detector area is sensitive for radiation in said second wavelength sub-range and is configured to generate said second detector signal, and

a vital signs detector for detecting a vital sign from a combination of said first and second detector signals by computing the difference between said first and second detector signals.

In a further aspect of the present invention, there is provided a corresponding method for vital signs detection.

Preferred embodiments of the invention are defined in the dependent claims. It shall be understood that the claimed method has similar and/or identical preferred embodiments as the claimed system, in particular as defined in the dependent claims and as disclosed herein.

The present invention is based on the recognition that the spectrum of radiation emitted from an illumination unit having a limited emission spectrum (sometimes also referred to as single wavelength technique), such as a LED (e.g. an NIR, i.e. near infrared, LED) spreads along a central value. This recognition is exploited to define two wavelength sub-channels (also called pseudo-color-channels), in which respective radiation reflected from a skin area of the subject is reflected. These sub-channels exhibit different relative PPG-pulsatility, while they have an identical sensitivity for motion-induced intensity-variations. Thus, through a combination of the detector signals from the sub-channels the influence of motion can be eliminated and a vital sign can be reliably and accurately determined from the motion-free combination of the detector signals.

The present invention may not only be used in the automotive field, where illumination in the invisible light spectrum may be applied, but also outside the automotive field. For instance, it may become interesting for patient monitoring in hospitals. A drawback of the currently proposed broad-spectrum solutions is that it is difficult to make them insensitive to ambient light. With a (pseudo-) single wavelength (or limited wavelength) technique this is much easier, as the radiation detector (e.g. a camera) can be made blind for anything outside the narrow band. This suppresses ambient light considerably.

According to embodiments of the invention said radiation detector comprises at least two detector areas, wherein a first detector area is sensitive for radiation in said first wavelength sub-range and is configured to generate said first detector signal and a second detector area is sensitive for radiation in said second wavelength sub-range and is configured to generate said second detector signal. Thus, by use of the detector areas the two detector signals in the different wavelength sub-ranges can be directly and simultaneously acquired. The radiation detector may, for instance, comprise an array of a plurality of first and second detector areas, in particular detector pixels, and may be configured as camera, e.g. RGB camera.

In a preferred embodiment said radiation detector comprises a first filter arranged for filtering incident radiation before being received by the first detector area and a second filter arranged for filtering incident radiation before being received by the second detector area, said first filter being configured for allowing radiation in said first wavelength sub-range to pass and said second filter being configured for allowing radiation in said second wavelength sub-range to pass. Using a radiation detector, e.g. a camera, with such a filter pattern, e.g. a Bayer filter pattern, that makes pixels more or less selective for wavelengths above or below the central value, easily creates two pseudo-color-channels (i.e. wavelength sub-ranges).

Preferably, in an optional configuration said first wavelength sub-range covers the lower half of said limited wavelength range and said second wavelength sub-range covers the upper half of said limited wavelength range. Thus, the wavelength sub-ranges substantially have the same bandwidth which balances the signal strength of the detector signals.

There are various options available for detection of vital signals from the detector signals. According to embodiments of the invention, said vital signs detector is configured to detect a vital sign by computing the difference between said first and second detector signals. Advantageously, the detector signals may be temporally normalized first, or their logarithm may be taken first. Alternatively, their ratio may be computed. However, there are a number of further options available. For instance, if the relative strength is exactly the same, a temporal normalization can be avoided. In all other cases a logarithm or a temporal normalization may be used.

Generally, a PPG signal results from variations of the blood volume in the skin. Hence the variations give a characteristic pulsatility “signature” when viewed in different spectral components of the reflected/transmitted light. This “signature is basically resulting as the contrast (difference) of the absorption spectra of the blood and that of the blood-less skin tissue. If the detector, e.g. a camera or sensor, has a discrete number of color channels, each with a different spectral sensitivity, e.g. each sensing a particular part of the light spectrum, then the relative normalized pulsatilities, i.e. the ratio of the relative pulsatilities, in these channels can be arranged in a “signature vector”, also referred to as the “normalized blood-volume vector”, Pbv. It has been shown G. de Haan and A. van Leest, “Improved motion robustness of remote-PPG by using the blood volume pulse signature”, Physiol. Meas. 35 1913, 2014, which is herein incorporated by reference, that if this signature vector is known then a motion-robust pulse signal extraction on the basis of the color channels and the signature vector is possible. For the quality of the pulse signal it is essential though that the signature is correct, as otherwise the known methods mixes noise into the output pulse signal in order to achieve the prescribed correlation of the pulse vector with the normalized color channels as indicated by the signature vector. Details of the Pbv method and the use of the normalized blood volume vector (called “predetermined index element having a set orientation indicative of a reference physiological information”) have also been described in US 2013/271591 A1, which details are also herein incorporated by reference.

There exist several known methods besides Pbv to obtain a pulse signal S from (normalized) detection signals, said methods being referred to as ICA, PCA, CHROM, and ICA/PCA guided by Pbv/CHROM, which have also been described in the above cited paper of de Haan and van Leest. These methods can be interpreted as providing the pulse signal as a mixture of different wavelength channels, e.g. red, green and blue signals from a color video camera, but they differ in the way to determine the optimal weighting scheme. In these methods the resulting weights are aimed at a mixture in which the distortions disappear, i.e. the “weighting vector” is substantially orthogonal to the main distortions usually caused by subject motion and/or illumination variations.

According to embodiments of the present invention detector signals can be obtained, which may subsequently be used to determine one or more vital signs. For instance, a standard RGB camera with an NIR-blocking filter removed (as radiation detector) may be used in combination with a single light source, such as an LED (as radiation source). This creates a highly cost-attractive option for obtaining the detector signals.

The radiation source may be configured to emit radiation in said limited wavelength range around a wavelength peak and the radiation detector may further comprise a peak filter for suppressing the peak wavelength. Alternatively, such peak suppression filter may also be comprised in the radiation source, although this may reduce the radiation energy sensed by the detector. This increases the difference in relative pulsatility (due to the PPG signal) of the two wavelengths that are sensed by both wavelength sub-channels and, hence, provides more discriminative power to distinguish motion (which always has the same relative strength in the two channels) and PPG signals, i.e. further improves the motion-robust detection of vital signs.

In a further embodiment said radiation source is configured to flash at a detection rate of the radiation detector at a duty cycle and said radiation detector is configured to integrate radiation detected during said duty cycle. This further reduces the ambient light sensitivity of the system.

In a practical implementation, particularly for automotive application or for application at night time, said radiation source is configured to emit radiation in a limited wavelength range around approximately 850 nm and said radiation detector is configured to detect radiation in a limited wavelength range around approximately 850 nm.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a device and of a system according to the present invention,

FIG. 2 shows a diagram illustrating the relative PPG amplitude over wavelength,

FIG. 3 shows a diagram illustrating the limited emission spectrum of an infrared LED,

FIG. 4 shows another embodiment of a radiation detector according to the present invention,

FIG. 5 shows a filter arrangement for use with a radiation detector according to the present invention, and

FIG. 6 shows a diagram illustrating the response of a conventional RGB camera.

### DETAILED DESCRIPTION OF THE INVENTION

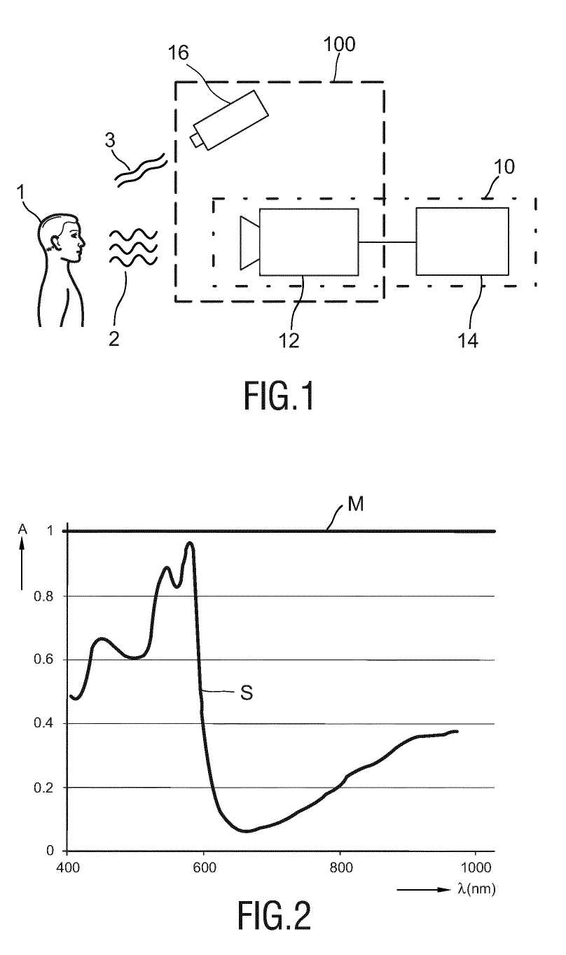

FIG. 1 shows a schematic diagram of a first embodiment of a device 10 and a system 100 according to the present invention. The device 10 comprises a radiation detector 12 for detecting radiation 2 reflected from a skin area of a subject 1, such as a patient, and for generating first and second detector signals from the detected radiation. The device 10 further comprises a vital signs detector 14 for detecting a vital sign (e.g. heart rate, SpO2, respiration rate, etc.) from a combination of said first and second detector signals.

The radiation detector 12 may e.g. be implemented as a photodetector or a camera, e.g. an RGB camera (optionally with an appropriate filter) and is configured to detect electromagnetic radiation from a skin area (e.g. the forehead, the cheeks, the hand, etc.) that is illuminated by radiation 3 of a limited wavelength range, e.g. by a radiation source 16, such as an LED (e.g. a near-infrared LED). The first detector signal generated by the radiation detector 12 represents radiation reflected from the skin area of a subject in a first wavelength sub-range of said limited wavelength range of radiation and the second detector signal represents radiation in a second wavelength sub-range of said limited wavelength range of radiation different from said first wavelength sub-range.

The vital signs detector 14 may e.g. be implemented in soft- and/or hardware, e.g. by a programmed computer or processor. Vital signs detection from such detection signals by use of remote photo-plethysmography is generally known in the art and shall not be further explained here. According to the present invention a combination of the first and second detection signals is made, from which the desired vital sign is then derived. For instance, the difference is determined between the first and second detection signals, i.e. time-variant detection signals are subtracted from each other (at each sampling time the values of the detection signals are subtracted). Other options of combinations include methods known as Pbv, ICA, PCA, CHROM, and ICA/PCA guided by Pbv/CHROM, as described in the above cited documents.

In the first embodiment the radiation detector 12 and the vital signs detector 14 together form the device 10, which may be implemented as separate elements or as a combined apparatus, e.g. as a camera that detects the radiation and processes the detection signals. The radiation source 16 and the radiation detector 14 form the system 100.

FIG. 2 shows a diagram illustrating the relative PPG amplitude A over wavelength λ. As shown in FIG. 2, the PPG-spectrum S is not completely flat. Using a steeper part of the spectrum, e.g. around 600 nm, would be preferred, but automotive applications require invisible illumination for night-time use, and also some medical applications, e.g. during the night, may require the use of invisible illumination. Since the PPG-spectrum S is hardly anywhere flat this is possible.

In contrast, as also shown in FIG. 2, a relative motion signal M reflecting motion of the subject 1 (and/or of the radiation detector 12 and/or of the radiation source 16, as shown in FIG. 1) does not depend on wavelength, assuming a homogeneous illumination spectrum.

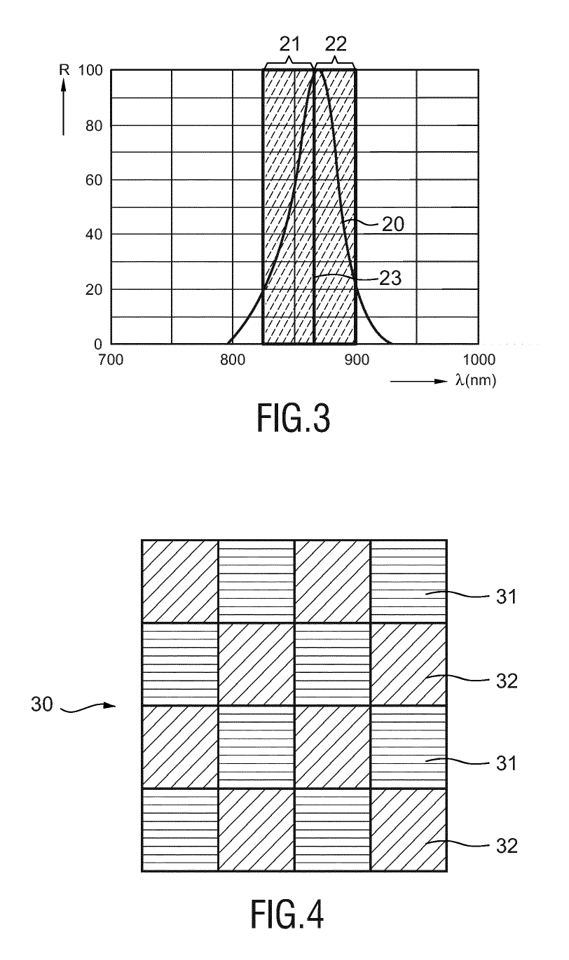

Consequently, in one embodiment, an LED with arbitrary NIR wavelength is used as radiation source 16 to illuminate the subject 1. FIG. 3 shows a diagram illustrating the limited emission spectrum 20 of an exemplary NIR LED (i.e. the relative radiant output R over the wavelength λ), which can be used in automotive applications and is substantially invisible for the driver. A camera, used as radiation detector 12 (as shown in FIG. 1), is pointed at the driver, e.g. at his/her face. As shown in FIG. 3 the exemplary NIR LED emits light with an emission spectrum 20 that has a central peak 23 just above 850 nm, with furthermore very substantially sub-range 21 and sub-range 22, each with a width of about several tens of nanometers, representing the lower half and the upper half of the emitted wavelength spectrum, respectively, i.e. the lower half covering the lower part of the wavelength spectrum with the lower frequencies and the upper half covering the upper part of the wavelength spectrum with the higher frequencies.

In an embodiment, illustrated in FIG. 4 as a front view, the radiation detector 30 comprises at least two detector areas 31, 32 (indicated by different hatching in FIG. 4), wherein a first detector area 31 is sensitive for radiation in said first wavelength sub-range and is configured to generate said first detector signal and a second detector area 32 is sensitive for radiation in said second wavelength sub-range and is configured to generate said second detector signal. Preferably, the radiation detector 30, e.g. an image sensor of a camera, comprises an array of a plurality of first and second detector areas, in particular detector pixels, wherein the single pixels or pixel groups represent the two detector areas 31, 32.

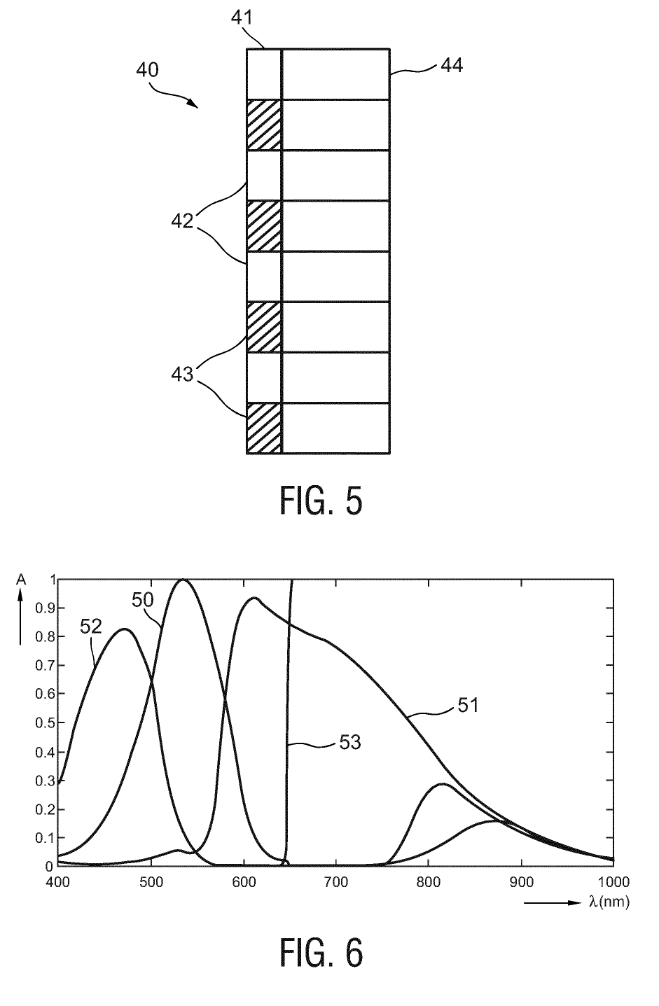

In another embodiment, illustrated in FIG. 5, the radiation detector 40, e.g. a camera, is equipped with a checkerboard pattern 41 of two different filters 42, 43, e.g. in front of the image sensor 44, as illustrated in FIG. 5 as a side view. The first filter 42 substantially passes a first wavelength sub-range 21, e.g. in this embodiment the lower half of the emitted wavelength spectrum 20, and the second filter 43 passes a second wavelength sub-range 22, e.g. in this embodiment substantially the upper half of the emitted wavelength spectrum 20, as illustrated in FIG. 3. Because the PPG-amplitude is higher for longer wavelengths (as shown in FIG. 2), the pixels with the second filter 43 will exhibit a higher relative PPG-signal, while the motion-induced noise signal components are identical in both channels.

The filters 42, 43 may be arranged alternately in front of individual pixels or pixel group of the radiation detector. Each pixel or pixel group may then provide a separate detector signals, which may then be grouped together (e.g. summed up or averaged) per filter to obtain a combined detector signal per type of filter.

In an alternative embodiment, the filters used are not very selective they only have a slightly different shape of their passband. This small difference can already cause sufficiently large relative pulsatility differences in the two resulting pseudo-color channels (and, thus, in the two detector signals) to distinguish PPG from motion. Sharper filters will yield a better SNR, but cheaper filters may be sufficient for a robust estimate of the pulse-rate.

In another embodiment an NIR radiation source (having an emission spectrum as shown in FIG. 3) is combined with a regular color video camera having an RGB-Bayer pattern with a spectrum as shown in FIG. 6. FIG. 6 particularly shows the relative response R over wavelength λ for the green channel 50, red channel 51 and blue channel 52 of an RGB camera. Further, the spectrum 53 of a visible light filter is shown. In this case, the blue and the green channels 52, 50 may act as the first and second filter, respectively. The red channel 51 may not be very different from the blue channel 52 and could be combined with the blue channel 52 which makes the number of pixels in both channels identical (green pixels occur twice as much as the red and blue ones in a Bayer pattern).

In a further advantageous embodiment, the camera (i.e. the radiation detector) may be equipped with a filter that blocks at least the visible light, i.e. having a spectrum 53 as shown in FIG. 6. This improves robustness for ambient light which is commonly obtained by flashing the LED (i.e. the radiation source) very briefly and exposing the camera only during these short bursts.

In another preferred embodiment, the visible light blocking filter may even take the shape of a band-pass filter that encompasses only the wavelengths emitted by the radiation source. This further improves robustness against ambient light.

A further improvement may result if additionally the light at the central peak (indicated as 23 in FIG. 3) in the emission spectrum 20 of the LED (i.e. radiation source) is blocked. Such blocking of the peak of the emission spectrum may alternatively be placed at the emission side, i.e. integrated with or close to the radiation emitter. This reduces the strength of the wavelengths that are sensed by both first and second filters, and hence provides more discriminative power to distinguish motion and PPG signals.

In further embodiments, the radiation source is flashing at the picture rate of the camera with a short duty cycle, while the camera integrates the light during said short duty cycle only to reduce the ambient light sensitivity of the system. Further, the narrow (limited) wavelength interval (and maybe other parameters, like the duty cycle of the flashing light) may be determined by the requirements of an automotive application with which the system is integrated.

As mentioned above, in some embodiments the motion and PPG signals may be separated with blind source separation means, like PCA or ICA.

In further embodiments the known relative pulsatility in the pseudo-color channels may be used to compute the pulse signal as a linear combination of the color channels, as e.g. described in the above cited publication of G. de Haan and A. van Leest.

The present invention may advantageously be applied in vital signs monitoring for automotive applications, e.g. for early detection of sleepiness, tiredness, risk of falling asleep, etc. Other applications are in the field of unobtrusive patient monitoring. The proposed invention may make such a device, system and method more robust to varying ambient illumination, and a single wavelength technique could become highly relevant as the camera can be blinded for most of the ambient spectrum.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A system for vital signs detection, said system comprising:
a radiation source for emitting radiation in a limited wavelength range for illuminating a skin area of a subject,
a radiation detector for detecting radiation reflected from a skin area of a subject in response to said illumination, and for generating first and second detector signals, the first detector signal representing radiation reflected from the skin area of a subject in a first wavelength sub-range of said limited wavelength range of radiation and the second detector signal representing radiation in a second wavelength sub-range of said limited wavelength range of radiation different from said first wavelength sub-range, wherein said radiation detector comprises at least two detector areas, wherein a first detector area is sensitive for radiation in said first wavelength sub-range and is configured to generate said first detector signal and a second detector area is sensitive for radiation in said second wavelength sub-range and is configured to generate said second detector signal, wherein said radiation detector comprises a camera, and
a vital signs detector for detecting a vital sign from a combination of said first and second detector signals by subtracting said first and second detector signals from each other.

2. The system as claimed in claim 1,
wherein said radiation detector comprises an array of a plurality of first and second detector areas, in particular detector pixels.

3. The system as claimed in claim 1,
wherein said radiation detector comprises a first filter arranged for filtering incident radiation before being received by the first detector area and a second filter arranged for filtering incident radiation before being received by the second detector area, said first filter being configured for allowing radiation in said first wavelength sub-range to pass and said second filter being configured for allowing radiation in said second wavelength sub-range to pass.

4. The system as claimed in claim 1,
wherein said first wavelength sub-range covers the lower half of said limited wavelength range and said second wavelength sub-range covers the upper half of said limited wavelength range.

5. (canceled)

6. The system as claimed in claim 1,
wherein said radiation source comprises a light source, in particular an LED.

7. The system as claimed in claim 6,
wherein said radiation source is configured to emit radiation in said limited wavelength range around a wavelength peak and wherein said radiation detector and/or said radiation source further comprises a peak filter for suppressing the peak wavelength.

8. The system as claimed in claim 1,
wherein said radiation source is configured to flash at a detection rate of the radiation detector at a duty cycle and wherein said radiation detector is configured to integrate radiation detected during said duty cycle.

9. The system as claimed in claim 1,
wherein said radiation source is configured to emit radiation in a limited wavelength range around 850 nm and said radiation detector is configured to detect radiation in a limited wavelength range around 850 nm.

10. A method for vital signs detection, said method comprising:
emitting radiation in a limited wavelength range for illuminating a skin area of a subject,
detecting by a radiation detector radiation reflected from a skin area of a subject in response to said illumination, wherein said radiation detector comprises a camera,
generating first and second detector signals, the first detector signal representing radiation reflected from the skin area of a subject in a first wavelength sub-range of said limited wavelength range of radiation and the second detector signal representing radiation in a second wavelength sub-range of said limited wavelength range of radiation different from said first wavelength sub-range, wherein said radiation detector comprises at least two detector areas, wherein a first detector area is sensitive for radiation in said first wavelength sub-range and is configured to generate said first detector signal and a second detector area is sensitive for radiation in said second wavelength sub-range and is configured to generate said second detector signal, and
detecting a vital sign from a combination of said first and second detector signals by subtracting said first and second detector signals from each other.
